(19) United States
(12) Patent Application Publication
(10) Pub. No.: US 2009/0099540 A1
(43) Pub. Date: Apr. 16, 2009

(54) ABSORBENT CORE FOR USE IN A DISPOSABLE WEARING ARTICLE

(76) Inventor: Fong Wah Sin, Sungai Petani (MY)

Correspondence Address: SCHMEISER OLSEN & WATTS, 18 E UNIVERSITY DRIVE, SUITE # 101, MESA, AZ, 85201, US

(21) Appl. No.: 11/873,297

(22) Filed: Oct. 16, 2007

(51) Int. Cl. A61F 13/15

(52) U.S. Cl. 604/368; 604/378; 604/385.01

(57) ABSTRACT

An absorbent core for use in a disposable wearing article for absorbing of liquid body exudates in humans and preventing leakage from the article is provided. The absorption of the liquid body exudates is facilitated by use of two different super absorbent polymers having different absorption under load and capable of forming a gel on contact with liquid.

### BACKGROUND OF THE INVENTION

1. Technical Field

The present invention relates to an absorbent core for use in a disposable wearing article contoured to be positioned against and enclose the crotch portion of a user, for absorbing and locking wetness and liquid body wastes such as urine.

2. State of the Art

There has been a large amount of activity in the field of disposable wearing articles such as baby diapers for absorbing and locking wetness and liquid body wastes such as urine. The prior art discloses several patents on baby diapers as well as diapers having super absorbent polymers.

The U.S. Pat. No. 6,306,121 issued in favor of Damaghi et al discloses an invention wherein is provided an absorbent article for use as a diaper or adult incontinence brief. The invention also focuses on use of elastic bands as securing means.

The U.S. Pat. No. 6,440,116 issued in favor of Tanji et al discloses an invention wherein a disposable absorbent article for containment of body wastes is provided. The invention further describes the method for containment of both liquid and semi solid human wastes.

The U.S. Pat. No. 6,579,273 issued in favor of Dupuy discloses a baby diaper in which a reusable absorbent insert is provided.

The U.S. Pat. No. 6,888,045 issued in favor of Wahlstroom et al discloses an invention wherein a method for producing a fibrous material is provided, and the said fibrous material is used for making a variety of absorbent articles such as diapers, incontinence guards, pant diapers and wound dressings etc.

The European patents EP-A-0287388 and FR-A-2586558 describe mechanisms to accomplish simplified change of diaper or incontinence guards by means of deploying variable attachment means.

The current scene in the technology today is flooded with absorbent articles for use by children. The existing prior art reveals that though they represent an improvement and advancement over the pre-existing technology, yet they are unable to provide a solution to the some of the following problems:

1) There is liquid seepage from margins of the wearing article;

2) The super absorbent polymer is spread on the surface of the absorbent article when it is used in a high quantity and such SAP on the surface sticks to the skin of the user and causes rashes and irritation.

3) There is clump formation in the middle of the wearing article on account of liquid absorption.

### SUMMARY OF THE INVENTION

The object of the present invention is to provide an absorbent core for use in wearing article equipped with two different super absorbent polymers having different absorption under load to avoid leakage from the article and provide enhanced comfort to the user.

An embodiment of the invention provides an absorbent core for use in a disposable wearing article, comprising a first super absorbent polymer having low absorption under load, placed on margins of the absorbent core; and a second super absorbent polymer having high absorption under load, placed on middle portion of the absorbent core and surrounded by the first super absorbent polymer from two sides of the margins; wherein when liquid is poured on the absorbent core, the liquid comes in contact with the second super absorbent polymer and part of the liquid flows to the first super absorbent polymer and forms a gel on contact with the first super absorbent polymer and said gel acts as a barrier to prevent leakage from the wearing article and to push the liquid back towards the second super absorbent polymer.

In another embodiment of the invention, the super absorbent material is in powder form.

For a better understanding of the present invention and to show how it may be performed, it will now be described in more details with respect to the accompanying drawings.

### BRIEF DESCRIPTION OF THE DRAWINGS

FIG. 1 is an overview of the absorbent core.

### DETAILED DESCRIPTION OF EMBODIMENTS OF THE INVENTION

The exemplary embodiments described herein in detail for illustrative purposes are subject to numerous variations. It is understood that various omissions, substitutions or equivalents are contemplated as circumstances may suggest or render expedient, but is intended to cover the application or implementation without departing from the spirit or scope of the invention.

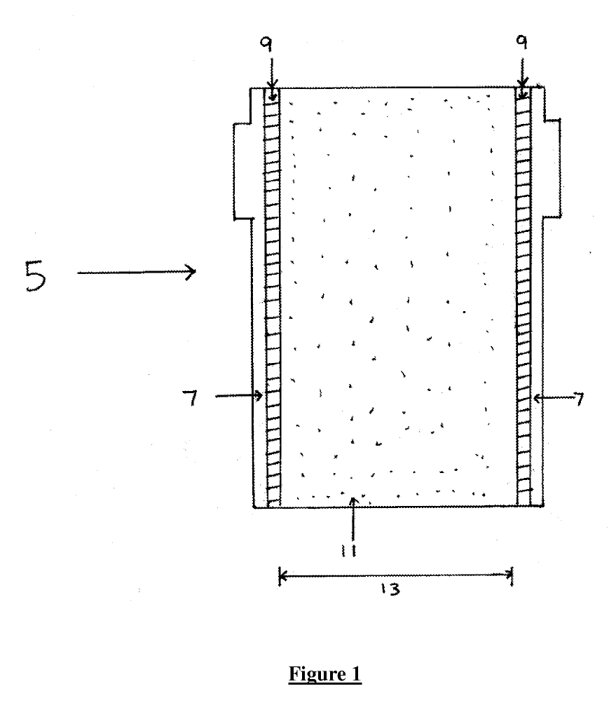

FIG. 1 is an overview of an embodiment of the absorbent core (5) of the present invention for use in disposable wearing article. The absorbent core (5) comprises of Middle Portion (13) and Margins (7). A first super absorbent polymer (9) is placed on the margins (7) of the absorbent core (5) and a second super absorbent polymer (11) is placed on the middle portion (13) of the absorbent core (5).

In a working mode of the embodiment of the present invention, the first super absorbent polymer (9) has a low absorption under load, which makes it capable of faster absorption. On the other hand, the second super absorbent polymer (11) has a high absorption under load, which makes it slower in absorption. Due to contrast in absorption properties of first (9) and second (11) super absorbent polymer, when liquid is pored onto the absorbent core (5), the second super absorbent polymer (11) does not absorb much liquid due to its slow absorption rate. Therefore, the liquid travels further towards the margins (7) from the middle portion (13) and comes in contact with the first super absorbent polymer (9). The first super absorbent polymer (9), owing to its fast absorption rate, reacts much faster than the second super absorbent polymer (11) and turns into a gel on coming in contact with liquid.

The gel formed by contact of liquid with first super absorbent polymer (9) grows to form various gel bands, which together act as a barrier located on the margins of the absorbent core (5). This barrier prevents leakage from the absorbent core (5) and also pushes any liquid coming towards the margins (7) from the middle portion (13), back towards the middle portion (13). This push-back by the barrier gives repeated opportunities and more time to the slow absorbing second super absorbent polymer (11) to absorb the liquid and thus gets rid of need of excessive or high usage of super absorbent polymer in the middle portion.

The invention further ensures that the absorption of wetness and liquid is enhanced by use of two super absorbent polymers with different absorption rates and qualities.

The disposable wearing article arrayed in the present invention is likely to find applications in arena of baby diapers, sanitary pads, disposable absorbent clothing, training pants, feminine care products, adult incontinence products, disposable boxer shorts, or the like.

Although, a particular exemplary embodiment(s) of the invention has been disclosed in detail for illustrative purposes, it will be recognized to those skilled in the art that numerous variations or modifications of the disclosed invention, including the variations in configuration as well as assemblage used in the embodiment, are possible.

Accordingly, the invention is intended to embrace all such alterations, modifications and variations as may fall within the spirit and scope of the forth coming claims.

I/We claim:

1. An absorbent core for use in a disposable wearing article, comprising:
a first super absorbent polymer having low absorption under load, placed on margins of the absorbent core;
a second super absorbent polymer having high absorption under load, placed on middle portion of the absorbent core and surrounded by the first super absorbent polymer from two sides on the margins;
wherein when liquid is poured on the absorbent core, the liquid comes in contact with the second super absorbent polymer and portion of said liquid is absorbed by the second super absorbent polymer and remaining portion of the liquid flows to the first super absorbent polymer and forms a gel on contact with the first super absorbent polymer and said gel acts as a barrier to prevent leakage from the wearing article and to push any outgoing liquid back towards the second super absorbent polymer for absorption thereof.

2. The absorbent core as claimed in claim 1, wherein the super absorbent polymer is in powder form.
